(19) United States
(12) Patent Application Publication
(10) Pub. No.: US 2002/0029069 A1
(43) Pub. Date: Mar. 7, 2002

(54) METHOD AND DEVICE TO MODIFY THE EXCITABILITY OF THE NEURAL NETWORKS

(76) Inventors: ROBERTO DELI, ROME (IT); GUIDO MARIA FILIPPI, VALLERANO (IT)

Correspondence Address: BEVERIDGE DEGRANDI WEILACHER & YOUNG, 1850 M STREET NW, SUITE 800, WASHINGTON, DC, 20036

(21) Appl. No.: 09/091,664

(22) PCT Filed: Dec. 20, 1996
(86) PCT No.: PCT/IT96/00261

(30) Foreign Application Priority Data
Dec. 22, 1995 (IT) RM.95-A/000844

(51) Int. Cl. A61N 1/00

(52) U.S. Cl. 607/48; 607/2

(57) ABSTRACT

The invention relates to a method to modify the excitability of the neuronal networks, comprising the application of vibration inducing muscular lengthening-shortening sequences with frequencies within the range of 1 and 300 Hz, with peak to peak motion amplitude included within the range from 1¼ and the maximum physiological length of the muscle, with an application time between 1 and 60 minutes. The invention also relates to the apparatus for performing the above mentioned method.

The present invention relates a method to modify the excitability of the neural networks.

Furthermore, the invention relates to an apparatus for the realisation of said method.

More particularly, the invention concerns a method and the relevant apparatus allowing to increase or to reduce the muscle tone and the contractile capability of whole muscle groups or of selected parts, even minimum, of a single muscle applying short sequences of micro muscle lengthenings—shortenings.

The method according to the invention allows to activate the mechanical nervous receptors which activate the motor neurones and the linked neural networks, thus increasing or reducing their excitability.

Such neuronal changes occurs at a motive level with remarkable modifications of the muscular activity.

By applications lasting few minutes each day, modifications of the muscular contractility, easily repeatable and reinforceable, occurs, said modifications lasting for long periods (months).

As it is well known, almost all the cellular structures are memory systems. Particularly, this is true for the excitable tissues (muscular and nervous), wherein the condition is a highly not linear function of the temporary conditions.

It has been demonstrated that it is possible to modify in vitro the memory of single cells of populations of cells, by intracellular injection of electrical charges lasting few minutes. Charges having high intensities produce an increased neuronal excitability for months, while low values of the charge determine persisting depressions of the neuronal excitability.

Nothing similar can be applied to the human being.

At present, contractility and muscular tone can be present increased o reduced by many different techniques.

Increase of the skeletal muscular tone are wished both in the rehabilitation and in the sport fields by the use of active motions, electrical stimulations, heat application, light, magnetic fields. Among these techniques, the most efficient are deemed to be those based upon the voluntary motion.

In the rehabilitation field, examples of these methods are the Kabat method, the Bobath method, the Doman-delacato method, the Vojta method, the progressive sequential method, methods based upon the use of the “biofeedback”.

These methods can be connected with those usually employed for the muscular strengthening in the agonistic and amateur sport fields. As a consequence of these methods, it is possible to obtain in a different way satisfying increase of the muscular trophism, increases of the neuromuscular reflexes. adaptation of articulation and bone charges, increase of the muscular strength.

However, as it is well known, the above methods require remarkable application time (often many hours each week for many months) and, in most cases, a high energetic expenditure.

Other methods presently known are characterised by a lower efficiency and applicability.

It is further used the electrostimulation, the action of this method being limited to the muscular fibres closer to the cutaneous planes upon which the electrodes are placed, due to its physical limitations. The application of the electrostimulation involves during the therapeutic process almost only the phasic fibres which, almost according to a rule, occupy this surface seat in the muscular contest.

Said fibres, due to their energetic metabolism, can be easily tired following to a long activity and cannot perform postural functions. To demonstrate this fact, it can be cited that it is necessary to limit the electrostimulation to 10-15 minutes each time.

Muscular modifications producing satisfying myo-relaxations are still more complex to be satisfying obtained during the execution and in any case with aleatory results.

In these cases, in view of the limited results obtained by the motion, the botulinum, the toxicity of which is well known, is used.

In this contest it is included the solution proposed according to the present invention allowing to completely modify the approach to the problems relevant to the correction of the skeletal muscular contractility persistently modifying the memory of little or big and selected neuronal populations, thus producing myo-relaxing actions or inducing great increases of the muscular force.

These and other results are obtained according to the present invention applying a technique providing the use of vibrations to impose muscular micro lengthening—shortening sequences for very short periods, with immediate response time, and a high selection of the muscular fibres to subject to the work.

The solution suggested according to the invention allows to act on the central neurone modifying the neuronal memory.

By the method and the apparatus according to the present invention it is possible to reach from the external, in a totally dry way, the neuronal network to be modified, using the natural sensorial ways physiologically provided and that are normally connected with the neuronal network controlling the muscular activity.

It is therefore specific object of the present invention a method to modify the excitability of the neuronal networks, comprising the application of vibration inducing muscular lengthening—shortening sequences with frequencies within the range of 1 and 300 Hz, with peak to peak motion amplitude included within the range from 1μ and the maximum physiological length of the muscle, with an application time between 1 and 60 minutes.

Particularly, according to the invention, one or more application each day are performed, and therefore maintenance sessions progressively less frequent.

Preferably, according to the invention, vibrations between 40 and 300 Hz, still more preferably between 80 and 120 Hz, and still more preferably of 90 Hz, are applied to obtain an increase of the force and of the muscular tone.

Furthermore, according to the invention, vibrations between 1 and 40 Hz, still more preferably between 10 and 30 Hz, and still more preferably of 15 Hz, are applied to obtain a satisfying muscular relaxation.

Always according to the invention, said peak to peak motion amplitudes are included between 1μ and 0,3×Lₒ (wherein Lₒ represents the average value between the maximum and minimum length of the muscular fibres involved), preferably between 0,1 and 2 mm, still more preferably between 0,1 and 1 mm.

Always according to the invention, said vibrations are applied employing pulse forces having a sinusoidal, square wave, saw tooth, triangular, etc. time run.

Preferably, said pulse forces will have a sinusoidal run.

The invention also relates to an apparatus allowing to modify the excitability of the neuronal networks, comprising a feeder, an amplifier, a mechanical transducer, means for generating vibration able to impose muscular lengthening—shortening sequences with frequencies within the range of 1 and 300 Hz and amplitude included within the range from 1μ and the maximum physiological length of the muscle, at one applying element for the connection of said means for generating vibrations with the specific body portion, and means to couple said vibration generation means with said applying element with respect to any body portion with any orientation with respect to the x, y and z axis and within a predetermined volume.

Particularly, according to the invention, frequency varies between 1 and 40 Hz, still more preferably between 10 and 30 Hz, and still more preferably of 15 Hz, to obtain a muscular relaxation, and between 40 and 300 Hz, still more preferably between 80 and 120 Hz, and still more preferably of 90 Hz, to obtain an increase of the force and of the muscular tone.

Still according to the invention, the apparatus allows to apply sequences with a peak to peak motion amplitudes between 1μ and 0,3×Lₒ (wherein Lₒ represents the average value between the maximum and minimum length of the muscular fibres involved), preferably between 0,1 and 2 mm, still more preferably between 0,1 and 1 mm.

Preferably, according to the invention, a wave generator is provided.

Particularly, said wave generator creates sinusoidal waves, square waves, saw tooth waves, triangular waves, etc.

Still according to the invention, said apparatus can comprise a voltage, frequency and amplitude of the applied force display as well as a timer.

According to a preferred embodiment of the apparatus according to the invention, said coupling means can be comprised of a vertical stand and of a bracket coupled to the same.

Furthermore, according to the invention, said applying element can be fixedly or removably coupled to said vibration generator.

The muscular micro lengthening—shortening can be obtained according to the invention applying pulse forces provided with components perpendicular with respect to the muscle fibres or by applying pulse forces pushing and pulling with a component parallel with respect to the muscle fibres.

The apparatus according to the invention can be used for example in a gymnasium, laboratory, surgery, at home, etc.

The present invention will be now described, for illustrative but not limitative purposes, according to its preferred embodiments, with particular reference to the figures of the enclosed drawings, wherein:

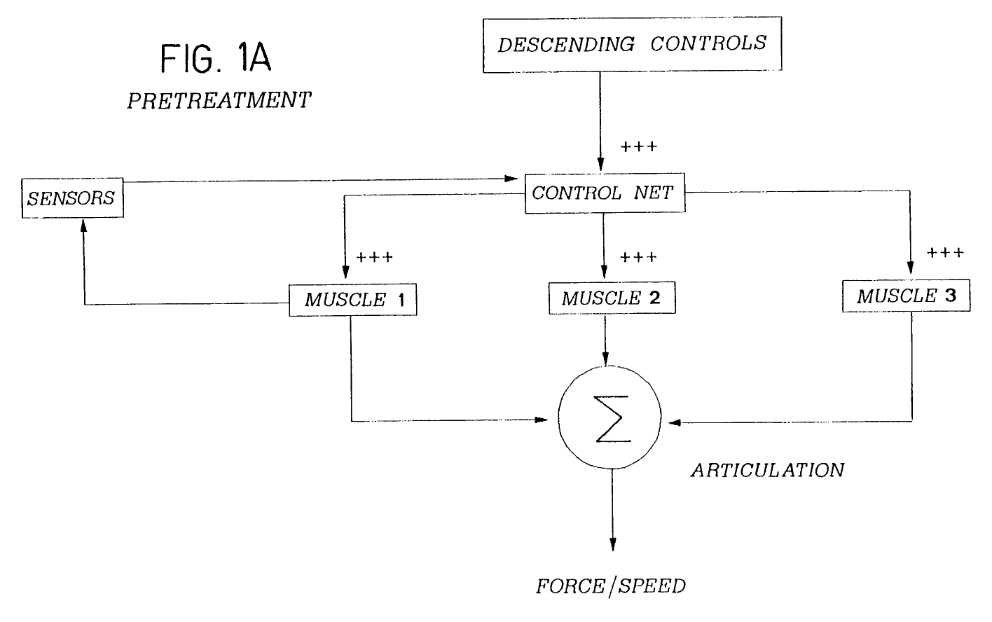

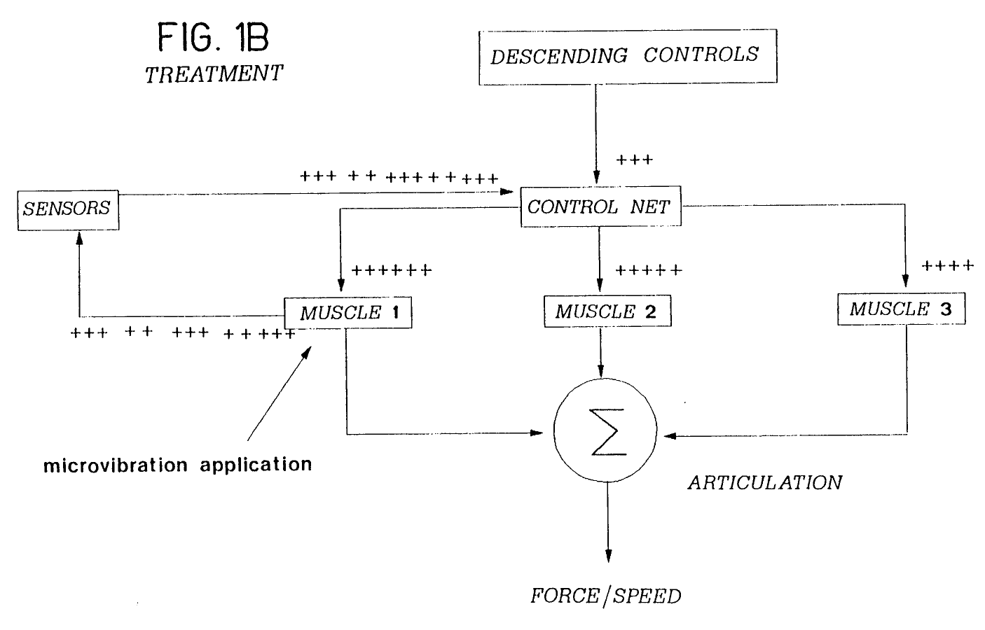

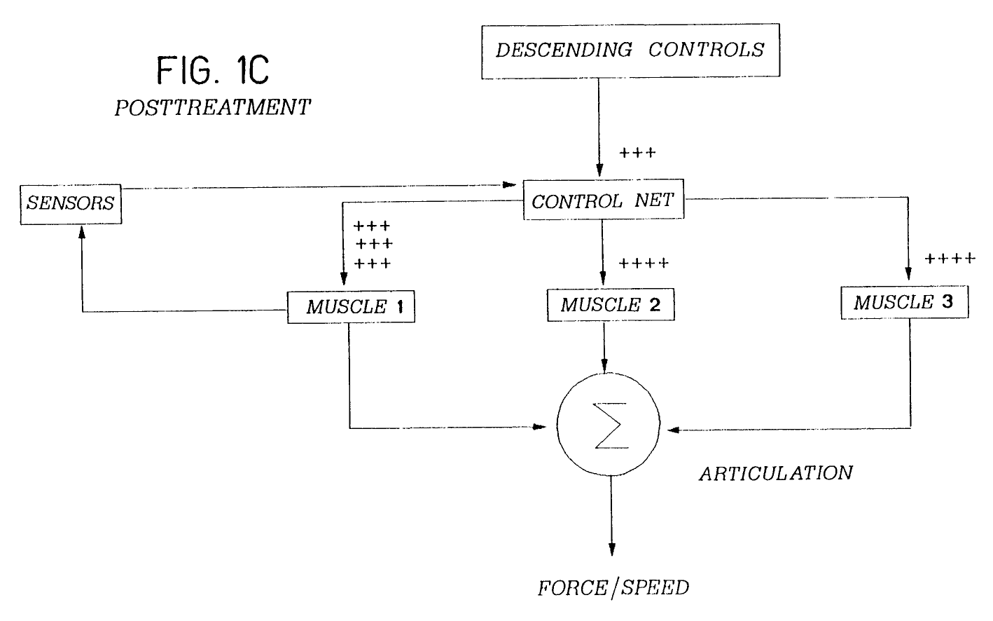

FIGS. 1 A, B and C are a scheme of an application of the method and of the apparatus according to the invention:

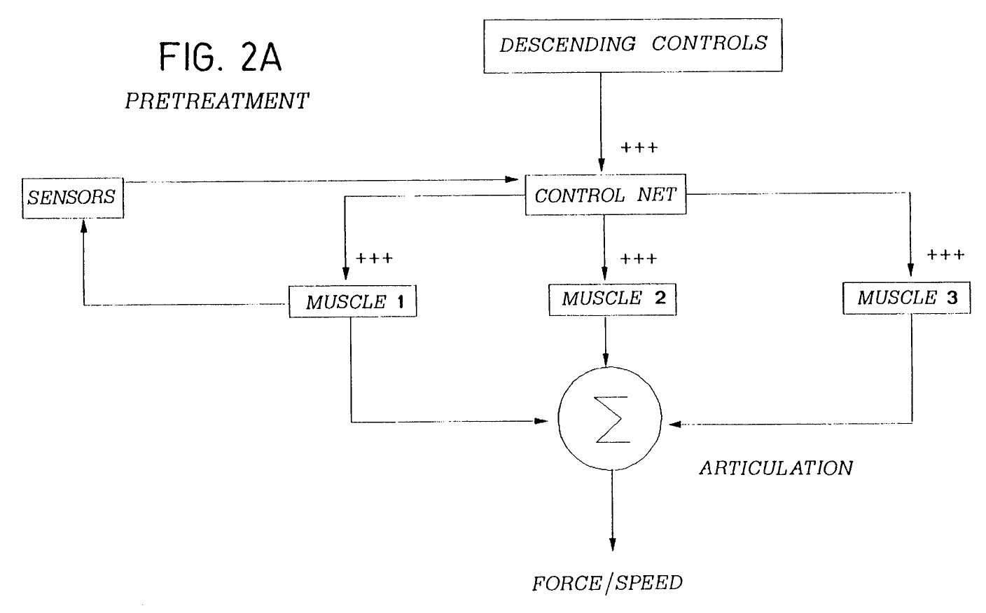

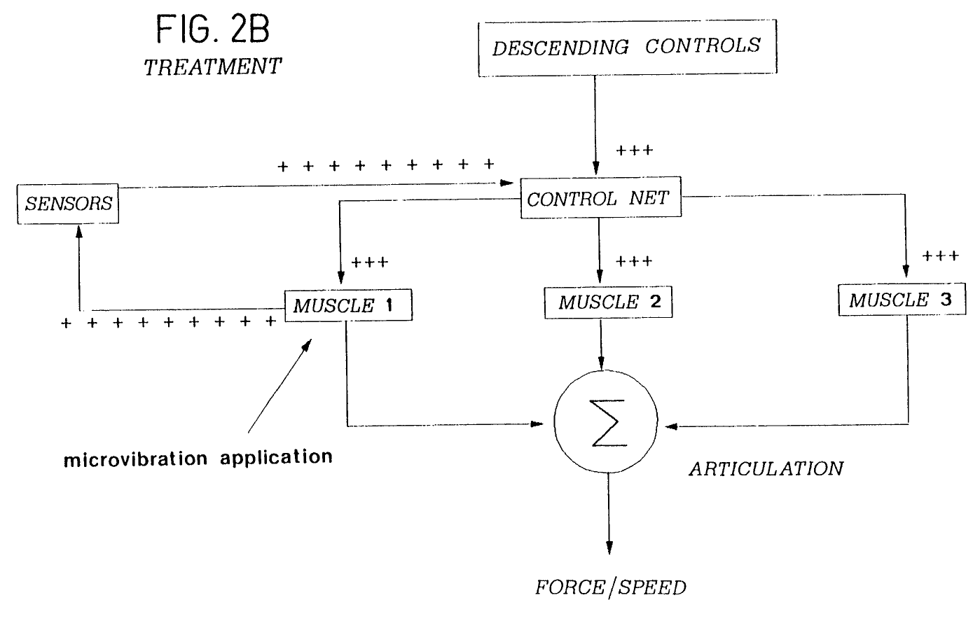

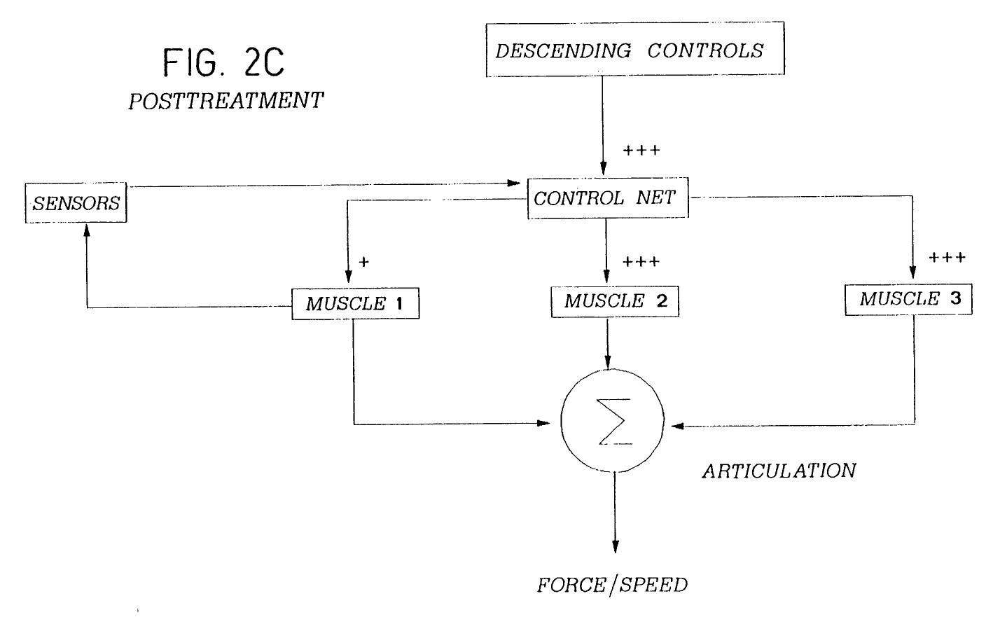

FIGS. 2 A, B and C are a scheme of a second application of the method and of the apparatus according to the invention;

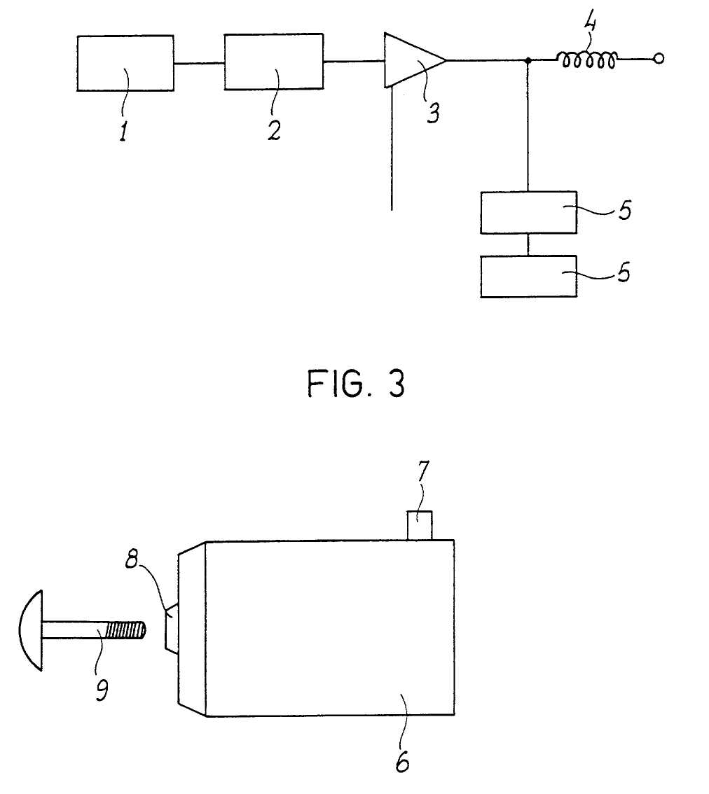

FIG. 3 diagramatically and partly by a block diagram shows an embodiment of the apparatus according to the invention;

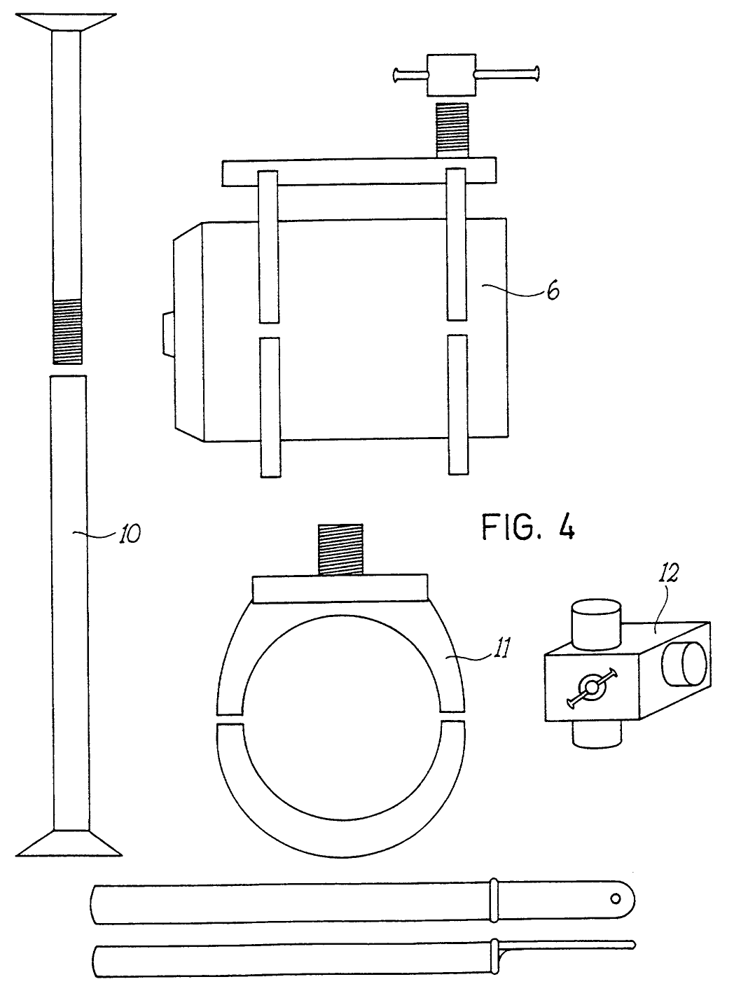

FIG. 4 diagramatically shows an embodiment of the apparatus according to the invention;

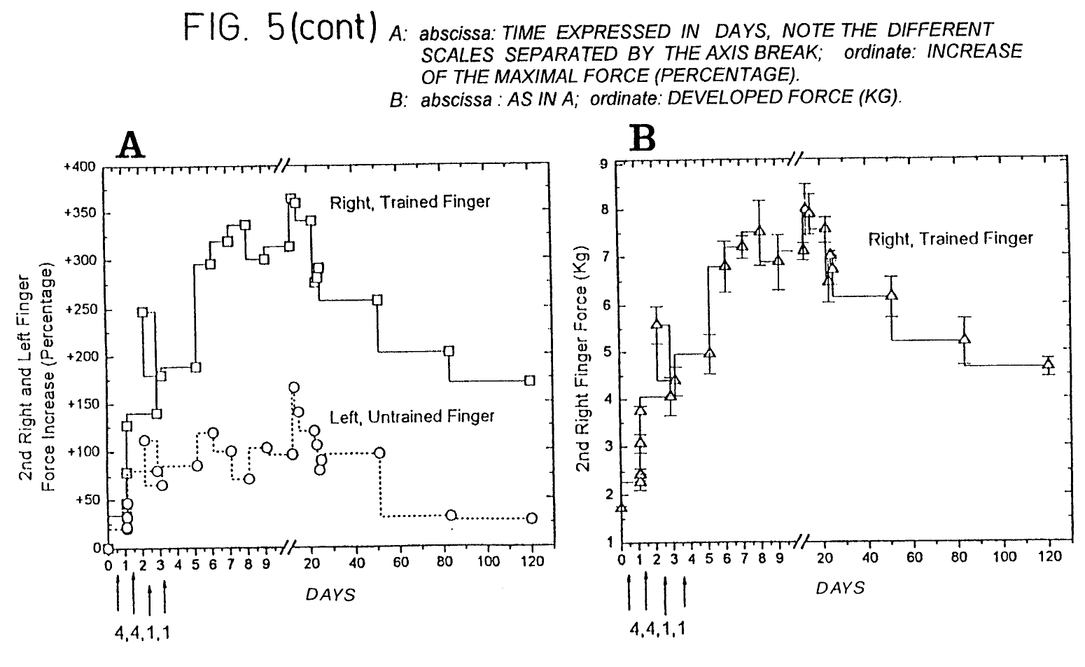

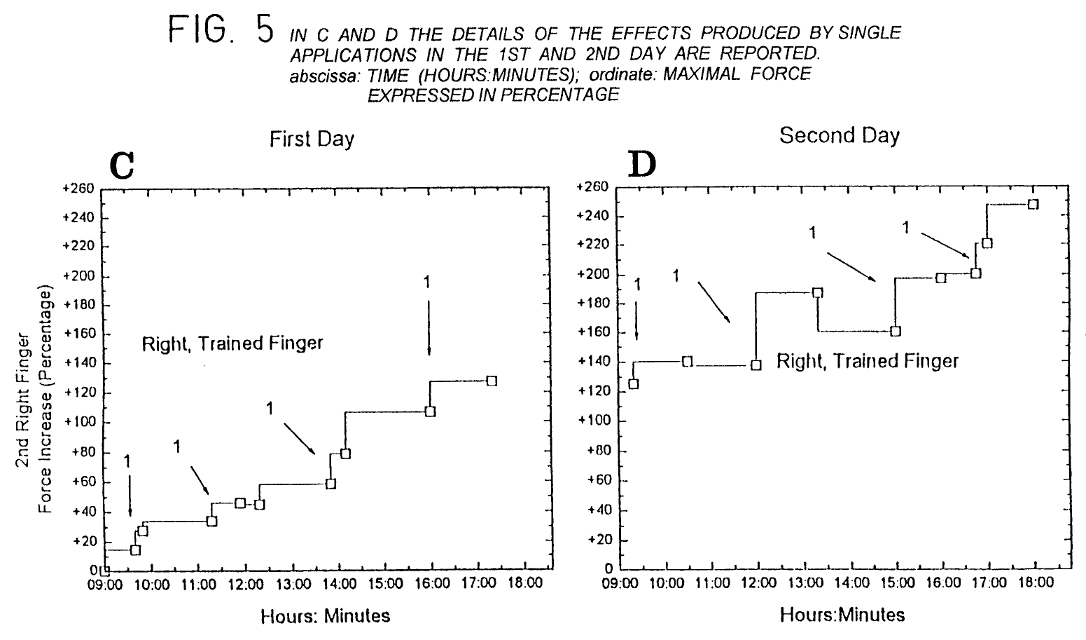

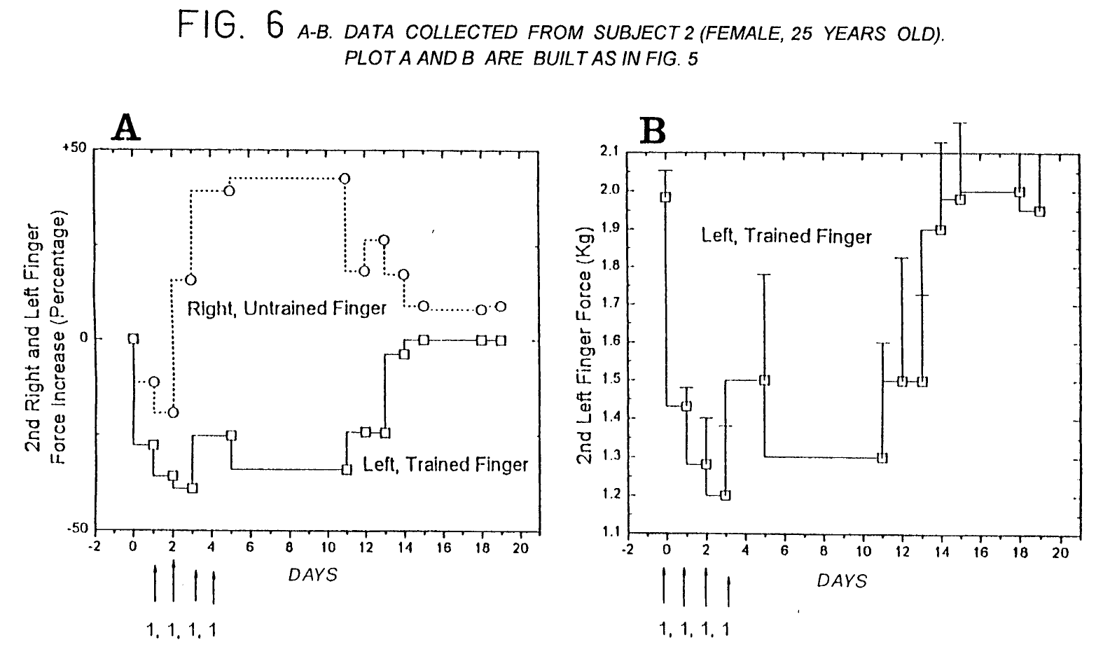

FIGS. 5 A-D show data collected during a first experiment;

FIGS. 6 A-B show data collected during a third experiment; and

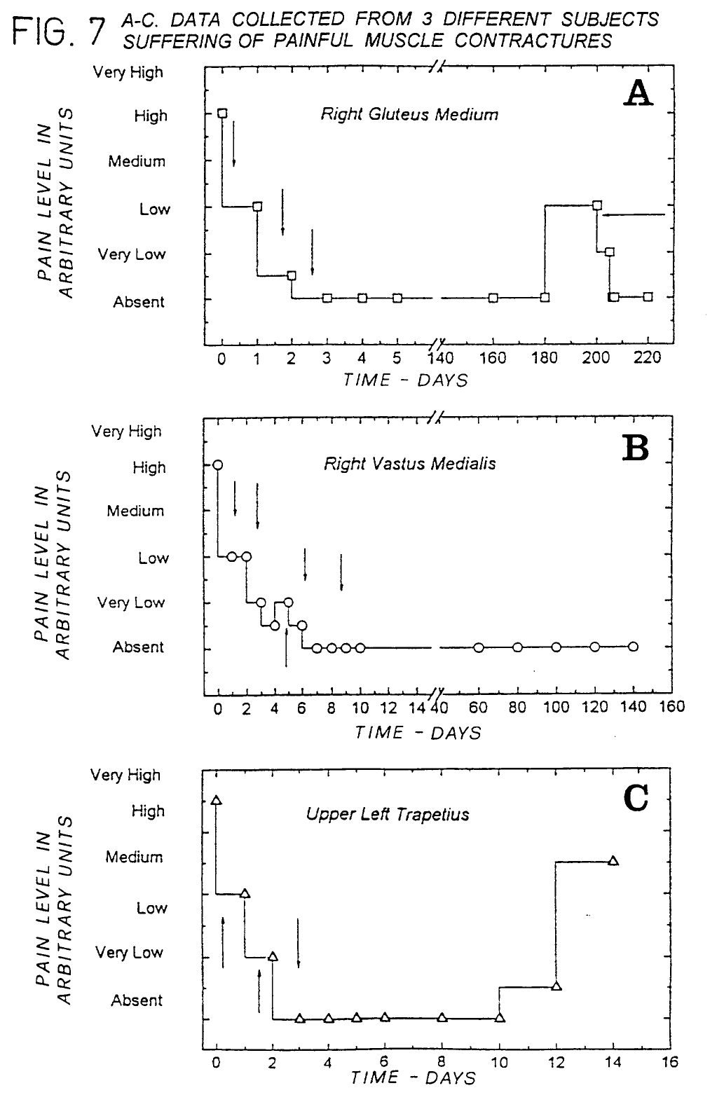

FIGS. 7 A-C show data collected during a fifth experiment.

What is proposed according to the invention is obtained applying muscular lengthening—shortening according to a continue succession by a mechanical transducer, controlled by a wave generator the output of which is suitably amplified, on whole or selected, even reduced, muscle groups, in function of the results to be obtained.

The amplitude of these movements must be such to generate a movement of the venter musculi, but must not be equal to or higher than values to induce injuries of the involved muscular fibres.

The frequency of the displacement applied must be chosen in function of the desired result: low frequencies will be advisable to obtain muscular relaxations, high frequencies will be suitable to obtain increase of force and muscular tone.

In fact, wishing to modify the muscular contractility, it is necessary to reach the motor segmental neural networks. Said structures physiologically receive commands having a remarkable power from the muscle mechanical neuro receptors (mechanical—electrical transducers). Such receptors are sensitive to mechanical micro deformations of their structure (μm order of magnitude) and to their derivatives.

Such deformation can be obtained by applying micro lenghtenings—shortenings of the muscle or of the muscular groups upon which it is desired to act. In fact, the nervous channels so activated are connected with the neural networks controlling the muscles wherein the same sensors are located (in other words they create control loops). Suitable frequencies and amplitudes of micro lenghtenings—shortenings sequences (the best results are obtained with oscillation frequencies of 80-150 Hz, peak to peak amplitude of 0,1-1 mm, and sinusoidal shape), generate a “paraphysiological” signal having a very high intensity on the connected neurons.

Such excitation, if maintained for suitable time (>5 minutes) induce a persisting increase of excitability of the networks, provide that said networks have been put in a slight voluntary activation during all the stimulus application time.

The application of these micro displacements (sinew, body part, etc.) one or more times each day allows to obtain as result after each application an increase of the neuronal excitability persisting for periods progressively longer after each applications, hours, days, months, up to a potential definitive condition.

The same procedure applied at low intensities (frequency 10-30 Hz, peak to peak amplitude 0,1-1 mm) induces a depression of the neuronal excitability, with features similar to what has been described in the above for the period of time of the applications necessary to obtain the persistency of the result.

By the apparatus according to the invention, a preferred but not limitative embodiment of which will be described in greater detail in the following, pulse forces are applied (to the muscular fibres, to the sinews, etc.) the run during the time of which can be with a sinusoidal, square. triangular, saw tooth wave, etc., piloted by a wave generator, amplified, generated by a transducer. It should be possible to modulate frequency and power of the signal. The transducer must be mechanically connectable to the human body, orientable in any direction within the space, firmly coupable to fixed support structures.

An embodiment of the apparatus according to the invention will be now shortly described making particular reference to the enclosed FIGS. 3 and 4, but it must be taken into consideration that the apparatus can be realised in different ways, always following the basic teachings of the present invention.

In the embodiment shown in the enclosed figures an instrument is shown providing a feeder 1, a wave generator 2, an amplifier 3 and a transducer 4.

Furthermore, displays 5 are provided.

The apparatus shown allows to adjust both the application amplitude and frequency of the vibrations, although such a feature is not essential for any solution. The apparatus according to the invention can allow to obtain only particular vibrations for particular applications.

The vibration generator 6 is provided with a feeding inlet 7, as well as with means 8 for coupling of the means 9 for applying the vibrations of the portion of the patient.

In FIG. 4 it is shown one of the different possible solutions to maintain the vibration generator 6 fixed according to any orientation along the three axis x, y and z and within a predetermined volume.

It is comprised of a support 10, adjustable in height, and of a bracket 11. By the adjustment element 12 it is possible to obtain the above mentioned orientation of the generator according to any possible orientation.

In this way, the apparatus according to the invention can be fixed according the direction of the longitudinal axis of any group of human muscular fibres with a non circular extension.

Said apparatus must and can have an angle between 0° and 180° with respect to the main axis of the muscular fibres with a generally circular extension, for example radial, circular, elliptical, so as to create cavitary structures, semi-cavitary structures, separation walls, sphincter structures.

Furthermore, said apparatus must and can be disposed tangential with respect to any trajectory with respect to a body portion.

The apical position of the apparatus allows the fixed coupling or a mechanical contact with the muscular fibres, sinews, body portions, imposing to the muscular fibres micro lenghtenings—shortenings sequences.

Obviously, the realisation of the fixed coupling of the apparatus according to the invention in such a way to be possible to obtain the above, can be realised in many different ways, all included within the scope of the present invention.

A first possible application of the method according to the invention and of the relevant apparatus is the induction of persisting increases of the force and of the muscular tone.

The application of muscular lengthening and shortening sequences having a suitable amplitude and frequency induces in the muscular fibres selected to be subjected to the treatment an increase of the force and of the muscular tone. These results are persisting and can be permanent with rare suitable reinforces of lengthening—shortening sequences.

To induce an increase of the force and of the muscular to persisting during the time it is necessary to follow the method described in the following and based on the use of high frequencies.

1) The muscular fibres upon which it is desired to act must be put in a light contraction condition. This contraction condition shall be maintained for all the duration of the session during which the muscular lengthening and shortening sequence is applied;

2) The frequency of the displacement imposed on the muscular structures will be between 40 and 300 Hz;

3) The amplitude of the displacements imposed to the muscular fibres will be, peak to peak, between 1μ and the maximum physiological amplitude of the muscle, preferably between 0,1 mm and 0,3 mm×Lₒ (wherein Lₒ represents the average value between the maximum and minimum length of the involved muscular fibres), still more preferably between 0,1 and 2 and still more preferably between 0,1 and 1 mm. High values of applied frequency will be coupled with low values of the amplitude;

4) The duration of each session will be in the range of magnitude of the minutes, and in any case not able to create a tiring feeling for the patient;

5) The number of day sessions necessary to induce the desired result should be suitable for the patient. When a contraction level having a valid value will be obtained, it will be possible to pass to a maintenance phase of the new muscular tone level reached, delaying the sessions in function of the spontaneous decay of the phenomenon obtained.

A second possible application of the method and of the relevant apparatus according to the invention is the induction of muscular relaxations persisting during the time.

A persisting muscular relaxation can be obtained applying the following scheme, based on the use of low frequencies.

1) The frequency of the displacement imposed on the muscular structures will be between 1 and 40 Hz;

2) The amplitude of the displacements imposed to the muscular fibres will be, peak to peak, between 1 mm and 0,3 mm×Lₒ (wherein Lₒ represents the average value between the maximum and minimum length of the involved muscular fibres). High values of applied frequency will be coupled with low values of the amplitude;

3) The duration of each session will be in the range of magnitude of the minutes:

4) The number of day sessions necessary to induce the desired result should be suitable for the patient. When a contraction level having a valid value will be obtained, it will be possible to pass to a maintenance phase of the new muscular tone level reached, delaying the sessions in function of the spontaneous decay of the phenomenon obtained.

In both cases, the use protocols of the method and of the apparatus according to the invention described in the above, the suspension of the treatment can be applied at any phase; it can be started again in any moment according to the procedures described for a regular treatment start.

By the method and apparatus according to the invention it is also possible to go back to the muscular condition before the treatment.

The muscular effects that can be obtained by the two protocols can be annulled by applying the protocol opposite to the one employed: use of low frequency for the lengthening—shortening sequence to reduce or annul the increase of tone and force obtained, use of the higher frequencies to restore suitable levels of force and muscular tone.

The basic mechanism of the present invention, as already said is the persistent modification of the memory of selected neuronal populations.

Almost all the cellular structures are memory systems.

This is particularly true for excitable tissues (muscular and nervous tissues) where the activity at a determined time is a function of the system state and this is function of the chronologically preceding state.

In the past is has been demonstrated that it is possible to modify in vitro (Kandel et al.) the memories of single cells or of cellular populations by intracellular injections of electrical charges for suitable time and with suitable intensity. High charge intensities applied for some minutes produce months of increased excitability of neurons subjected to this treatment. Low charge intensities instead provoke a persisting neuronal hypoexcitability phenomenon. Similar phenomenons have been stressed by the same techniques of intracellular electrostimuiation for primordial animals (molluscs).

Nothing similar has been stressed or tested for in vivo mammal neurons, and least at all for human being.

The solution suggested according to the present invention is able to persistently modify the memory of selected neuronal populations, thus producing immediate effects of increase of the force and of the tone or of relaxations of the muscle structure upon which it is desired to intervene.

The method and the apparatus described allow to obtain the motion control neuronal networks by the activation of some selected sensitive pathway, which physiologically connect the muscle structures with the nervous networks controlling the same.

Said sensorial paths are selectively activated by applying sequences of muscular micro lenghtenings—shortenings, the amplitude and the following derivatives during the time of which are a powerful and highly selective stimulus (Matthews).

The activity of these sensitive paths, selected on the basis of their elective sensitivity to the kind of stimulus, is precisely and powerfully modulated by the frequency and amplitude parameters of the displacements imposed. Said activity of the sensorial paths is sent to the nervous network exerting the motion control of the muscular fibres involved by the mechanical stimulus.

Said fibres are selectively characterised by a very high resistance to the prolonged maximum activity (Burke).

In this completely physiological way, a situation similar to those artificially created in vitro and non injecting currents within the body of single neurons wherein microelectrodes are inserted.

The application of said high intensity mechanical stimulus is translated into a powerful inlet signal to the above mentioned networks, while less strong mechanical stimulus produce low inlet signals.

The two conditions, maintained for some minutes, provokes increase (see FIG. 1) or reduction (see FIG. 2) of the excitability of the activated neuronal networks, persisting for months and even more.

Neurons upon which one of the two state modifications has been produced will be inclined to pilot the muscular fibres with frequencies respectively higher and lower with respect to the preceding situation, thus producing higher force output or myorelaxations.

More generally, said mechanisms can be operative when stretch receptors connected with motion neurons are present, thus also in the smooth muscles and myocardial tissues.

The method and the apparatus according to the invention can be used in many fields, among which the following must not be interpreted in a limitative sense:

need of increasing or reducing the neuronal excitability, need of magnifying the obtainable muscular force, need of inducing myorelaxations, need of shaping the muscular tissue. Muscle powering, staying power increase. Modulation of the basic tone to modulate, fluidify, optimise, improve the motive strategy learning time. Modifications of the body posture or of parts of the body posture.

Muscle hypertonia, modification of the use of anomalous motive strategies, muscle cramp, muscular hyperreflexivity, modulation of the basic muscular tone to modulate, fluidify, optimise, improve the motive strategy learning time, muscle development in a direction of a composition for motive units having phasic features (high tension for short time, contraction speed, low staying power), modification of the body posture or of parts of the body posture.

The use of the method and of the apparatus according to the invention is not however limited to the muscles controlled by the will of the human, but it can be also used for muscles out of the voluntary control (plain muscles: intestine, arteries and veins, vesica, etc.; myocardial muscle: heart, etc.)

### EXPERIMENTS AND RESULTS

All the experiments reported were performed on 54 adult subjects who gave their informed consent.

The induced increases of the motor output are illustrated only by the experiments in which the patient subjective reports were not influential to the demonstration. Since the induced decreases of the motor output in painful muscle contractures can be poorly documented by objective results, subjective reports are presented.

Throughout all the duration of the experiments, the patients kept unchanged their way of life.

Clear and positive results have always been obtained. At the beginning of the experimental research, in 5 patients, in which it was wished to induce an increase of the motor output, the initially absence of immediate positive effects resulted to be due to the incapacity of the patients to produce a valid voluntary contraction in the treated muscles. In this cases the protocol started again after the subject had been trained to contract his muscles. In these 5 cases also it have been obtained results superimposible to the others for the time course and the magnitude of the effects.

In all the tested subjects no changes in the mass of the trained region were detected.

### EXPERIMENT 1

Aim: to increase the motor output of the flexor muscles of the 2nd right finger.

Methods: the force developed by the maximal voluntary contraction (MVC) of the flexor muscles of the 2nd right finger of 6 adult subjects was measured before and after the operative conditioning above described and consisting of 10 vibratory applications, the time duration of each being 10 minutes. The applications were devised according to the following sequence: 4+4+1+1 distributed over 4 consecutive days; an interval of at least 1 hour was elapsed between two consecutive applications.

Vibration parameters: Sinusoidal vibration; Frequency: 90 Hz; Amplitude 0.3 mm peak to peak.

Patient position during applications and during force evaluations: patient position was carefully kept the same in order to avoid any variable eventually produced by different positions.

The patient was seated. Occiput and back were kept in contact with a rigid chair back. The chair back was at an angle of 90° with the horizontal plane. Patient's arm and his hand were positioned as described below:

a (Lateral rotation of the elbow)=about 25°

b (Forward rotation of the elbow)=about 30°

g (Shoulder joint angle)=about 120°

d (Wrist joint angle)=about 180°

e (Metacarpophalangeal joint angle)=about 120°

θ(1st finger joint angle)=about 135°

η(2nd finger joint angle)=about 120°

The vibratory stimulus was directed along the same direction of the force vector, and the vibratory device was applied to the distal end of the 2nd finger.

No particular instruction was given concerning the left arm.

The tested patient exerted his MVC downward with his 2nd right finger against a force transducer. The subject started to exert his maximal force at an acoustic “go” signal and stopped when he felt to have developed his maximal effort, each trial lasted 5-9 sec. At least 2 minutes elapsed between two consecutive trials, to avoid possible post-tetanic and fatiguing effects. The value assumed as the control MVC force was obtained by averaging 90 single trials, divided in three blocks of 30 trials, a block a day during three consecutive days. During and after conditioning each MVC value was obtained by averaging 10 consecutive tension evaluations. All the recorded responses were measured by evaluating the peak tension.

Obtained Results

The time course and the entity of the effects were superimposible in all the tested subjects. The maximal force increase was observed 15 days after the end of the conditioning and it ranged between +335% and +370% (mean increase+351.25%±14). The increase of the MVC force decrease down to 50% of its maximum (T/2) after 115 days±10 from the beginning of the experiment. FIG. 5 A-D reports a typical data collection from 3 males 38 years old. In each plot, the points corresponding to time=0 (control values before operative conditioning are the average of 90 single evaluations performed during 3 days, 10 measures a day. All the other points are the average of 10 measures. Arrows and relative numbers indicate the application day and the number of applications. In plot B the standard deviation is reported. Plot 5A shows the entity of the increase (in percentage) of the maximal force developed by the 2nd right finger (open squares, solid line), which was trained as above described. In the same plot it is also reported the maximal force of the 2nd left finger which was not trained (open circles, dotted line). The first point at day 0 is the control value and it is the mean of 90 trials. All the successive points are the mean of 10 evaluations. Standard deviation is shown in plot B. The large increase of the developed force in the right finger and the very long persistence of the effect are clearly shown (the maximum, +365%, was reached after 16 days from the end of training and a force increase of +112% was still present after 116 days from the end of the training). An evident effect (plot A, open circles, dashed line) it was also detected in the left untrained finger. Such a result is in agreement with data reported by Wolpaw in primates (6) and might be attributed to the bilateral projections of Ia afferents and to an incomplete controlateral muscle relaxation. Plot B shows the same effect on the right finger; the magnitude is expressed in Kg. Finally, plot C and D show the action of the detailed action of the first eight applications, clearly evidencing the effect of each application.

In two subjects a short lasting sequence of oscillations (5 minutes) at low frequency (14 Hz) was applied to the same trained finger, 15 days after the end of the potentiating training. Immediately the force output returned to its control output.

No patient referred any discomfort or pain or any other alteration in the trained region.

### EXPERIMENT 2

Aim: to increase the muscle tone in the Vastus Medialis muscle.

Methods: the same protocol presented above, consisting in these experimental series of 8-15 applications, was applied to 5 adult subjects. All these patients were suffering of condromalacia of Rotula because of a lateral deviation of the rotula which was forced to touch the adjacient Tibia. All the patients were operated at least one year before, to reposition the Rectus Femoris tendon. The surgical operation was technically successful in all the patients but poorly satisfactory about the degree of Rotula deviation, since Vasts Medialis tonus was smaller than the Vastus Lateralis tone. The vibratory stimulation was applied while the patient was sitting and keeping the leg in hyperextension (contraction of the Vastus Medialis).

Vibratory stimulation was applied to the muscle body.

Obtained Results

All the patients obtained after the second application day a clear Rotula deviation to the correct position, a total relief of the pain symptomatology and a clear improvement of deambulation. After 7 months from the end of the treatment the position of the Rotula is still right and the symptoms still absent.

### EXPERIMENT 3

Aim: to decrease the motor output of the flexor muscles of the 2nd left finger.

Methods: the force developed by MVC of the flexor muscles of the 2nd left finger of 4 healthy adult subjects was measured before and after the operative conditioning above described and consisting of 10 vibratory applications, the time duration of each application was 10 minutes. The applications were devised according to the following sequence: 1+1+1+1 (one application a day).

Vibration parameters: Sinusoidal vibration; Frequency: 14 Hz; Amplitude 0.3 mm peak to peak. The experimental procedures were the same described in experiment 1. It was asked to the subject to exert a light force (100-180 g) against the vibrator.

Obtained Results

In all the tested subjects the reduction of the MVC force appeared about 30-60 minutes after the application. The average persistence of the phenomenon resulted to be of 8 days (±2) and the mean maximal reduction was of −48% (±12%). FIG. 6 shows the typical results collected in subject 3 (male, 34 years old). Note that in the controlateral finger it was observed an increase of the MVC force. It might be attributed to the repetitive isometric efforts performed by the subject to evaluate the results. Consequently, the same potentiating mechanism could mask the real entity of the decrease of the MVC force obtained in the 2nd left trained finger.

### EXPERIMENT 4

Aim: to increase the resistance to the muscle fatigue.

Methods: in 5 athletic subjects a single application was performed to the Biceps Brachii and to the Rectus Abdomenis. The time duration of the application was 10 minutes, the frequency was 90 Hz and the peak to peak oscillation was 0.1 mm. The applications were performed directly on the muscle bodies.

In the day after the application the subjects performed their sequence of exercises against constant weights. The increase in the number of exercises performed after the application was compared with the control.

Obtained Results

All the subjects referred an absence of muscle fatigue in the trained muscles and in the surrounding territories during their normal physical training. The increase in the number of performed exercises was +123%±13 in the first 4 days after the application. The maximum was reached in the 4 day (+138%±10) and decreased in the following 3 days down to the control.

### EXPERIMENT 5

Aim: to induce a decrease of the motor output in order to relief painful muscle contractures. A 39 patients group was tested. Their common symptom was a painful muscle contracture, which was clearly appreciable by palpation. The patients were divided in three main groups according the origin of their algic contracture, as defined on the base of patients' history and eventually confirmed by instrumental tests:

Contractures originating from eccentric contractures (23 subjects).

Contractures originating from abnormal postures (10 subjects).

Contractures originating from joint and bone diseases (6 subjects).

Methods: low frequency vibration (frequency 14 Hz; amplitude 0.3-0.5 mm; application time: 5 minutes) was applied 1-5 times, one time a day, on the point in which muscle contracture was very well appreciated by palpation. Such a point generally corresponded to the most painful region.

Obtained Results

Muscle contractures produced by eccentric contractures or by abnormal body postures were promptly eliminated at the end of the treatment; first results generally appeared about 3-5 hours after the first applications. The effects persisted for a period ranging from three to eight months (178 days±34 being the average unpainful period).

Even if muscle contractures produced by abnormal postures were relaxed with the same fast time course as those produced by eccentric contractures, the persistence of the results largely depended on the way of life of the subject, since a large part of these syndromes (70%) were due to professional postures.

Muscle contractures originating from joint or/and bone diseases were generally improved within shorter periods: 10-20 days. The exception to such results was represented by the situations in which bone and joint diseases produced reflexed muscle contractures generating abnormal postures. In this cases, the induced muscle relaxation appeared to act as a “reset”, which allowed the subject to find again the correct motor strategies and the persistence of the effects was the same as the above described two groups.

Three examples referred to the three groups of patients are illustrated in FIG. 7 A-C. Plot A shows the effects obtained in a subject (female, 27 years old) suffering from a 2 years persisting lomboalgia produced by the painful contracture of the right and left Gluteus Medium muscles. Since such muscles are very important in keeping the vertical posture, the subject's physical activity was largely limited. In this case 3 applications were performed and were sufficient to produce the pain relief. Physical activity was fully recovered and up to 175 days from the end of the therapy the subject kept an excellent physical condition. 197 days after the end of the first treatment 3 applications were repeated and the muscle relaxed again.

Plot B illustrates the case of a subject (male, 52 years old) who, 32 years before, reported a large number of bone fractures as a consequence of a fall from the 4th floor. He survived, but, as a consequence, he assumed an abnormal and complex vertical posture which produced very marked and painful muscle contractures particularly localised in right and left Trapetius, right and left Gluteus Medium and Quadriceps muscles, (mainly in Vastus Medialis muscle). The motility of the subject was largely impaired and painful. In this subject 5 applications, distributed during 5 days intervalled by 1 resting day, were imposed to each muscle, on the point in which the pain and the contracture were maximal; plot A reports the results obtained on the right Vastus Medialis muscle, being the results induced in the other muscles similar. At the moment, five months after the applications the subject asserts to feel only a slight increase of the muscle tone in the left Vastus Medialis m.

Plot C shows results obtained by 3 applications performed on a patient suffering from painful muscle contractures extended to the right and left neck muscles and to the masticatory muscles. The origin of which was likely due to the presence of a marked cervical arthrosis. As reported in plot C, the relief persisted only 14 days following the end of the 4th application.

### DISCUSSION ON THE RESULTS OF THE EXPERIMENTS

The reported results show that it is possible to obtain large and persisting changes of motor output in man, by a rapid and non invasive technique, consisting of an externally imposed muscle vibration at high (to obtain the potentiation of the motor output) or at low (to obtain the decrease of the motor output) frequency, superimposed on an isometric muscle contraction. Such a muscle contraction was voluntarily produced by the subjects or induced by clinical diseases. The obtained changes of motor output can have their expression in:

large and long lasting increases of the maximal developed force (experiment 1);

changes of the body posture (experiments 2);

large and long lasting increases of the muscle fatigue resistance (experiment 3);

large and persistent decreases of motor output (experiment 4);

relief of painful muscle contractures (experiment 5).

All the effects were obtained by selectively activating (16, 21, 22, 24) the same afferent pathway, Ia afferent fibres, used by other authors to show that spinal cord is a plastic substratum (6, 7, 8, 10, 15, 20, 28-35). The rapidity of the induction (30-60 minutes), the time course of the effect and the control of the dimensions of the trained muscle region allow to discard the possibility that such changes can be due to changes of the muscle mass.

The increased motor output showed three phases: induction, maintenance and extinction. The enhanced motor output was rapidly abolished by an adequate technique (experiment 1). Moreover potentiating effects resulted to increase the MVC force also after the end of the conditioning. This last datum is shown in FIG. 5 A in which a progressive increase of the motor output persisted up to 16 days after the end of the applied operative conditioning. Such long lasting increase was observed in all the other tested subjects and referred by themselves as subjective feeling. Actually, since normal movements involve, at the same time, descending commands and Ia afferent inflow (produced by gamma and beta coactivation (16)) to the same Alfa motoneurons, it could be suggested that the same operative conditioning, even if at lower levels, is continuously applied during normal life. Such a situation could counteract the normal time course of the operative conditioning effects (5, 23), increasing the duration of the persistence phase.

The induced decreases of motor output were characterised by a more rapid induction phase, followed by the phases of maintenance and extinction.

Some considerations can be forwarded with the aim to speculate on the underlying mechanisms activated by this technique.

Concerning the protocol employed to obtain an increase of the motor output, it can be considered a form of operative conditioning (5). Since the CS and the afferent pathways (Ia fibres) were similar to those employed on monkeys by other authors (6, 7, 15, 28-35), it seems reasonable to suggest that the involved mechanisms can be the same. Consequently the neural circuits affected by the conditioning are limited to spinal cord and they may be constituted of the Alfa-motoneurons and, perhaps, of the interneurons impinging on the Alfa-motoneurons (6, 7, 15, 28-35). Moreover the obtained effects show many features which are commonly observed in Long Term Potentiation (LTP). LTP is a well known form of enduring enhancement in synaptic transmission (27). As in LTP (26), in the present study it was observed that the time course showed by the induced increases of the motor output is composed of three phases: acquisition, maintenance, extinction. The potentiating effect appears after a few minutes from the end of the first conditioning, as observed in LTP induction (26). Finally, in analogy with LTP (13), the potentiating effect can be eliminated by means of a 4-6 minutes application of a weak form of the same stimulus. The same features allow us to discard the possibility of attributing the obtained increases of the motor output to post-tetanic potentiation (5).

Two other peculiarities, similar to the LTP properties, were clearly individuated in our results. The potentiating effect can be obtained only if the two stimuli are present at the same time (Associativity) (13). The potentiating effect is not present if only one input is furnished to the central neural networks (Cooperativity) (17). All these analogies suggest the possibility that the phenomenon which we elicited has many features which characteristically belong to LTP. Anyway, at the moment, we lack of the necessary intracellular data to demonstrate that this form of operative conditioning produces a true LTP.

The last groups of experiments (experiments 3 and 4) which we presented showed the possibility of reducing the motor output by applying to the subject a low frequency, weak stimulus for 1-5 minutes. Results reported on the flexor muscles of the left 2nd finger (experiment 3) are not impressive in magnitude and it contrasts with the subjective effects easily obtained in patients suffering of muscle contractures (experiment 4). However the measures of the reduction of the motor output was performed by evaluating the muscle force developed by MVC before and after operative conditioning. This procedure, even if it allows to perform objective evaluations, implies the application of the opposite protocol (applications of 90-100 Hz vibratory stimulation) above illustrated and discussed, many times a day. Actually, during each evaluation a strong Ia synaptic activity (16) and a maximal descending command cooperate in the Alfa motoneurons and likely on their interneurons. Consequently it can be inferred that the obtained decreases of the motor output were largely underestimate in their amplitude and time course. The results obtained in the 2nd right finger (not trained in experiment 3, see FIG. 4A) sustain this hypothesis.

The patients to whom this new therapy was applied obtained long lasting and impressive improvements of their painful and physically limiting condition. Also in these cases some hypothesis might be inferred concerning the underlying mechanisms. However the protocol which was applied to obtain motor output decreases is different from those applied in monkeys (6, 7, 15, 28-35). In the experimental conditions chosen by these authors, the level of the descending command (i.e. the magnitude of the muscle contraction) appears to be the most important variable. In our situation the peripheral stimulus characteristic is the determinant parameter. Consequently we cannot affirm a strict association between the two protocols. However, on the basis of our data it is possible to suggest some interpretation of the reported results. In 31 cases in which the muscle contracture started not later than 24 years before, a full and persistent relief was obtained only by 1-3 applications. It can be suggested that the motor output decrease could be due to a phenomena similar to that reported in experiment 2. In this experiment a weak and short application of the reinforcing stimulus (a low frequency vibration) can induce a sudden disappearance of the potentiating effect obtained by the previously applied operative conditioning. It could be suggested that the painful muscle contractures which resulted to be promptly relaxed by only 1-3 applications, could be originated by behavioural situations commonly indicated as eccentric contractions (i.e. muscle contractions on which a sudden and strong stretch is inscribed, as it often happens in car crushing, sport activities, etc.). These conditions are similar to those employed in our experiments (experiments 1 and 2). It is interesting to note that eccentric contractions can also be easily produced in states of abnormal postures, in which the physiological motor strategies are altered. Consequently, the effects produced by behavioural conditions, very similar to the ones produced by our operative conditioning, can easily vanish by only one or few applications of the same reinforcing stimulus at low intensity as reported in experiment 1. On the contrary, the cases in which more applications were requested (particularly in long lasting muscle contractures, more than linear than 10-20 years in some subjects) could be really seen as cases in which a true depression of the motor output was induced, by a protocol similar to the one used to determine the increase of the motor output. The latency and the time course of the effects lead us to infer that the obtained depression has some common features with another form synaptic plasticity, the Long-Term Depression (LTD) recently identified (1, 27). LTD is a form of plasticity characterised by a long lasting synaptic depression. LTD is considered the opposite form of LTP (1).

The possibility that the technique proposed in this study can alter the excitability of the Alfa motoneurons and/or of interneurons impinging on them, as indicated by the Wolpaw's group (6, 7, 15) and suggested by the analogies between the results reported in the present paper and the LTP and LTD effects could explain the obtained increases and decreases of the motor output. Actually, plastic changes of the excitability of the neurones determining the final motor command could likely alter the recruitment of new motor units and the motoneuron firing. These two variables are commonly indicated as the two factors by which the muscle force output is regulated (4).

The important increase of skeletal muscle resistance to the fatigue can be attributed to the powerful increase of Ia synaptic input to the alpha-motoneurons, since it was recently evidenced a determinant role of the γ-Ia circuit in the muscle fatigue.

The most important datum underlined by this research is that human neural circuits belonging to spinal cord can be largely and persistently modified by rapid and non invasive techniques. Since these techniques request the voluntary contraction of the muscle fibres controlled by the neurones on which it is necessary to act, the presented protocols allow to involve only one single motor units as well as large muscle territories. Spinal cord plasticity and the relative simple methods which allow to act on it disclose many new fields and really new perspectives to the therapeutic possibilities and to the sport medicine.

The present invention has been described for illustrative but not limitative purposes, according to its preferred embodiments, but it is to be understood that modifications and/or changes can be introduced by those skilled in the art without departing from the relevant scope as defined in the enclosed claims.

1. Method to modify the excitability of the neuronal networks, characterised in that it comprises the application of vibration inducing muscular lengthening—shortening sequences with frequencies within the range of 1 and 300 Hz, with peak to peak motion amplitude included within the range from 1μ and the maximum physiological length of the muscle, with an application time between 1 and 60 minutes.

2. Method according to claim 1, characterised in that one or more application each day are performed, and therefore maintenance sessions progressively less frequent.

3. Method according to claim 1 or 2, characterised in that vibrations between 40 and 300 Hz are applied to obtain an increase of the force and of the muscular tone.

4. Method according to claim 3, characterised in that vibrations between 80 and 120 Hz are applied.

5. Method according to claim 3, characterised in that vibrations of 90 Hz are applied.

6. Method according to claim 1 or 2, characterised in that vibrations between 1 and 40 Hz are applied to obtain a satisfying muscular relaxation.

7. Method according to claim 6, characterised in that vibrations, between 10 and 30 Hz are applied.

8. Method according to claim 6, characterised in that vibrations of 15 Hz are applied.

9. Method according to one of the preceding claims, characterised in that said peak to peak motion amplitudes are included between 1μ and 0,3×Lₒ (wherein Lₒ represents the average value between the maximum and minimum length of the muscular fibres involved).

10. Method according to claim 9, characterised in that said peak to peak motion amplitudes are included between 0,1 and 2 mm.

11. Method according to claim 9, characterised in that said peak to peak motion amplitudes are included between 0,1 and 1 mm.

12. Method according to one of the preceding claims, characterised in that said vibrations are applied employing pulse forces having a sinusoidal, square wave, saw tooth, triangular, etc. time run.

13. Method according to claim 12, characterised in that said pulse forces have a sinusoidal run.

14. Apparatus allowing to modify the excitability of the neuronal networks, characterised in that it comprises a feeder, an amplifier, a mechanical transducer, means for generating vibration able to impose muscular lengthening—shortening sequences with frequencies within the range of 1 and 300 Hz and amplitude included within the range from 1μ and the maximum physiological amplitude of the muscle, at one applying element for the connection of said means for generating vibrations with the specific body portion, and means to couple said vibration generation means with said applying element with respect to any body portion with any orientation with respect to the x, y and z axis and within a predetermined volume.

15. Apparatus according to claim 14, characterised in that the frequency varies between 1 and 40 Hz.

16. Apparatus according to claim 15, characterised in that the frequency varies between 10 and 30 Hz.

17. Apparatus according to claim 15, characterised in that the frequency is of 15 Hz.

18. Apparatus according to claim 14, characterised in that the frequency varies between 40 and 300 Hz.

19. Apparatus according to claim 18, characterised in that the frequency varies between 80 and 120 Hz.

20. Apparatus according to claim 18, characterised in that the frequency is of 90 Hz.

21. Apparatus according to one of the preceding claims 14-20, characterised in that at least a load cell is provided.

22. Apparatus according to one of the preceding claims 14-21, characterised in that sequences with a peak to peak motion amplitudes between 1μ and 0,3×Lₒ (wherein Lₒ represents the average value between the maximum and minimum length of the muscular fibres involved) are applied.

23. Apparatus according to claim 22, characterised in that preferably sequences with a peak to peak motion amplitudes between 0,1 and 2 mm are applied.

24. Apparatus according to claim 22, characterised in that preferably sequences with a peak to peak motion amplitudes between 0,1 and 1 mm are applied.

25. Apparatus according to one of the preceding claims 14-24, characterised in that a wave generator is provided.

26. Apparatus according to claim 25, characterised in that wave generator creates sinusoidal waves, square waves, saw tooth waves, triangular waves, etc.

27. Apparatus according to claim 26, characterised in that a sinusoidal wave is created.

28. Apparatus according to one of the preceding claims 14-27, characterised in that said apparatus comprises a voltage, frequency and amplitude of the applied force display as well as a timer.

29. Apparatus according to one of the preceding claims 14-28, characterised in that said coupling means are comprised of a vertical stand and of a bracket coupled to the same.

30. Apparatus according to one of the preceding claims 14-29, characterised in that said applying element can be fixedly or removably coupled to said vibration generator.

31. Apparatus according to one of the preceding claims 14-30, characterised in that the muscular micro lengthening—shortening is obtained applying pulse forces provided with components perpendicular with respect to the muscle fibres or by applying pulse forces pushing and pulling with a component parallel with respect to the muscle fibres.

32. Method to modify the excitability of the neuronal networks according to anyone of the preceding claims 1-13, substantially as illustrated and described.

33. Apparatus allowing to modify the excitability of the neuronal networks, according to anyone of the preceding claims 14-31 substantially as illustrated and described.
